(19) United States
(12) Patent Application Publication
(10) Pub. No.: US 2026/0102376 A1
(43) Pub. Date: Apr. 16, 2026

(54) USE OF DIHYDROTANSHINONE I DERIVATIVE IN PREPARATION OF DRUG FOR TREATING CEREBRAL CAVERNOUS MALFORMATION

(71) Applicant: Nanjing University of Chinese Medicine, Nanjing (CN)

(72) Inventors: Yang ZHAO, Nanjing (CN); Weiwei ZHENG, Nanjing (CN); Suyun YU, Nanjing (CN); Jia LI, Nanjing (CN); Yin LU, Nanjing (CN)

(73) Assignee: Nanjing University of Chinese Medicine, Nanjing (CN)

(21) Appl. No.: 19/286,267

(22) Filed: Jul. 31, 2025

(30) Foreign Application Priority Data
Oct. 14, 2024 (CN) 202411431855.X

(51) Int. Cl. A61K 31/4188; A61K 9/00; A61K 9/16; A61K 9/20; A61K 9/48; A61K 47/20; A61K 47/26; A61P 7/00

(52) U.S. Cl. CPC A61K 31/4188; A61K 9/0019; A61K 9/1652; A61K 9/2059; A61K 9/4866; A61K 47/20; A61K 47/26; A61P 7/00

(57) ABSTRACT

A use of a dihydrotanshinone I derivative in preparation of a drug for treating cerebral cavernous malformations (CCMs) is provided. The dihydrotanshinone I derivative can treat CCM with excellent efficacy. The dihydrotanshinone I derivative has a chemical name of (R)-1,6-dimethyl-11-phenyl-1,10-dihydro-2H-furo[2′,3′:1,2]phenanthro[3,4-d]imidazole and a molecular formula of C₂₅H₂₀ON₂. The research has shown that the dihydrotanshinone I derivative can play a prominent anti-CCM role in vivo. The dihydrotanshinone I derivative can remarkably reduce the lesion counts and volumes in CCM, and thus is suitable for the preparation of anti-CCM drugs.

### CROSS REFERENCE TO THE RELATED APPLICATIONS

This application is based upon and claims priority to Chinese Patent Application No. 202411431855.X, filed on Oct. 14, 2024, the entire contents of which are incorporated herein by reference.

### TECHNICAL FIELD

The present disclosure relates to a use of a dihydrotanshinone I derivative in preparation of a drug for treating a cerebral cavernous malformation (CCM) and belongs to the field of traditional Chinese medicine pharmaceuticals.

### BACKGROUND

CCMs are a common cerebrovascular disease with an incidence rate of approximately 0.5%. Patients with CCMs are clinically manifested with intracranial hemorrhage, stroke, epilepsy, or even sudden death. The pathological characteristics of CCMs include dilated lumens, thinned vessel walls, disrupted endothelial cell-to-cell junctions, lack of mature vascular wall components such as smooth muscle cells, accompanying thrombosis, etc. Currently, the treatment of CCMs primarily relies on surgical intervention, and there is a lack of effective medications, which has been highly valued by the Ministry of Health of the People's Republic of China.

Mice with the endothelial cell-specific knockout of the CCM 1 gene (Ccm1) (Ccm1^{ECKO}) exhibit distinct pathological manifestations of CCMs, which can provide a reliable animal model for drug development. Salvia miltiorrhiza, as one of the most common clinical drugs, can promote blood circulation and resolve blood stasis, and can also cool blood and alleviate inflammatory abscesses. Salvia miltiorrhiza demonstrates remarkable efficacy in treating cardiovascular diseases. The effective components of Salvia miltiorrhiza can be categorized into liposoluble components such as tanshinone, danshenol, and salviol and water-soluble components such as salvianic acid A and salvianolic acids. These effective components have various pharmacological effects including anti-platelet aggregation, reducing blood viscosity, alleviating inflammatory responses, dilating coronary arteries, and anti-fibrosis. Animal experiments have shown that the dihydrotanshinone I derivative ((R)-1,6-dimethyl-11-phenyl-1,10-dihydro-2H-furo[2′,3′:1,2]phenanthro[3,4-d]imidazole (DPDH)) exhibits significant therapeutic potential in ameliorating CCMs. There has been no research on the use of DPDH for treating CCMs.

### SUMMARY

An objective of the present disclosure is to provide a use of a dihydrotanshinone I derivative in preparation of a drug for treating CCM.

The drug for treating CCM involved in the present disclosure is a dihydrotanshinone I derivative with a chemical name as follows:

(R)-1,6-dimethyl-11-phenyl-1,10-dihydro-2H-furo[2′,3′:1,2]phenanthro[3,4-d]imidazole, a molecular formula of C₂₅H₂₀ON₂, and a structural formula as follows:

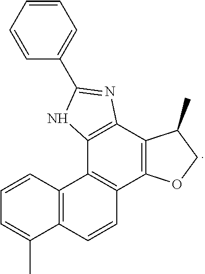 [SMILES: Cc1cccc2c1ccc1c3c(c4nc(-c5ccccc5)[nH]c4c12)[C@@H](C)CO3]

In the use of a dihydrotanshinone I derivative in preparation of a drug for treating CCM, the dihydrotanshinone I derivative and a pharmaceutically-acceptable adjuvant are prepared into one of an oral formulation and an injection.

In the use of a dihydrotanshinone I derivative in preparation of a drug for treating CCM, the oral formulation is one of a tablet, a hard capsule, a soft capsule, and a granule.

In the technical solutions of the present disclosure, with a Ccm JECKO mouse model, the overall pharmacological effect of DPDH in mice is investigated through stereo microscopy, hematoxylin and eosin (H&E) staining, computed tomography (CT) imaging, and scanning electron microscopy for brain tissues, so as to provide important experimental evidence for the preparation of anti-CCM drugs.

The experimental results of the present disclosure have shown that DPDH exhibits a significant effect for improving CCMs in vivo both in terms of reducing the lesion counts and volumes and alleviating the endothelial structure.

The dihydrotanshinone I derivative of the present disclosure, as an active pharmaceutical ingredient, may be prepared with a pharmaceutically-acceptable carrier and/or excipient into an anti-CCM drug in any dosage form suitable for humans or animals, including an oral formulation, an injection, and a tablet.

### BRIEF DESCRIPTION OF THE DRAWINGS

FIG. 1 shows a proton nuclear magnetic resonance (¹H NMR) spectrum of DPDH;

FIG. 2 shows a carbon-13 nuclear magnetic resonance (¹³C NMR) spectrum of DPDH;

FIGS. 3A-3B show the influence of DPDH on CCMs in Ccm1^{ECKO} mice, where FIG. 3A shows the stereo microscopy results and FIG. 3B shows the H&E staining results;

FIGS. 4A-4C show the influence of DPDH on the lesion counts and volumes for CCMs in (Ccm1^{ECKO} mice, where FIG. 4A shows the CT imaging results, FIG. 4B shows the quantification for the lesion counts, and FIG. 4C shows the quantification for the lesion volumes; and

FIG. 5 shows the influence of DPDH on vascular structures of CCMs in (Ccm1^{ECKO} mice.

### DETAILED DESCRIPTION OF THE EMBODIMENTS

The present disclosure will be further described below with reference to specific embodiments. It should be understood that these embodiments are only intended to describe the present disclosure, rather than to limit the scope of the present disclosure. In addition, it should be understood that various changes and modifications may be made to the present disclosure by those skilled in the art after reading the content of the present disclosure, and these equivalent forms also fall within the scope defined by the appended claims of the present disclosure.

The present disclosure proposes a potential anti-CCM drug for treating CCM. The present disclosure is further described in detail below in conjunction with the specific embodiments of the present disclosure. Unless otherwise specified, the raw materials and reagents adopted in the following examples all are commercially-available products, or may be prepared by known methods.
Example 1: 10 g of a dihydrotanshinone I derivative was taken, a starch was added, and tableting was conducted to produce a tablet.

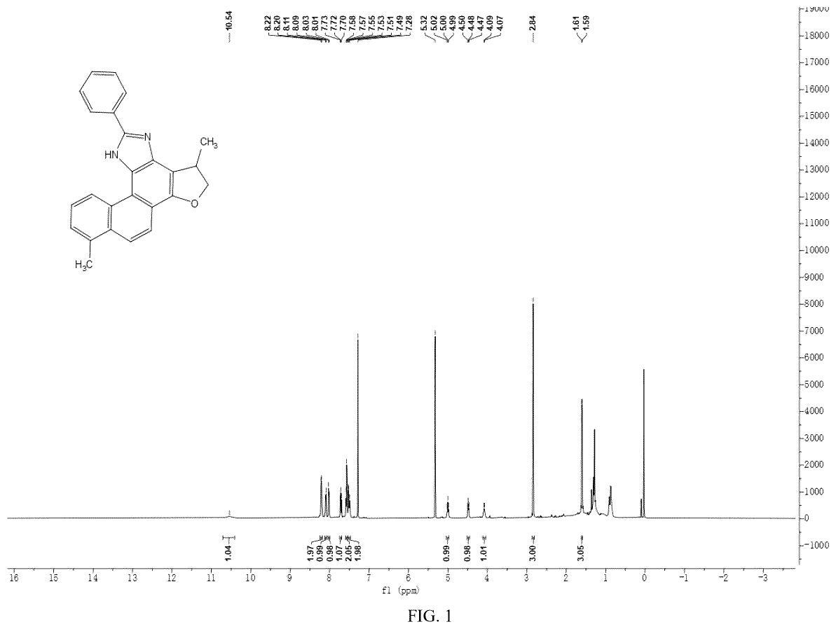

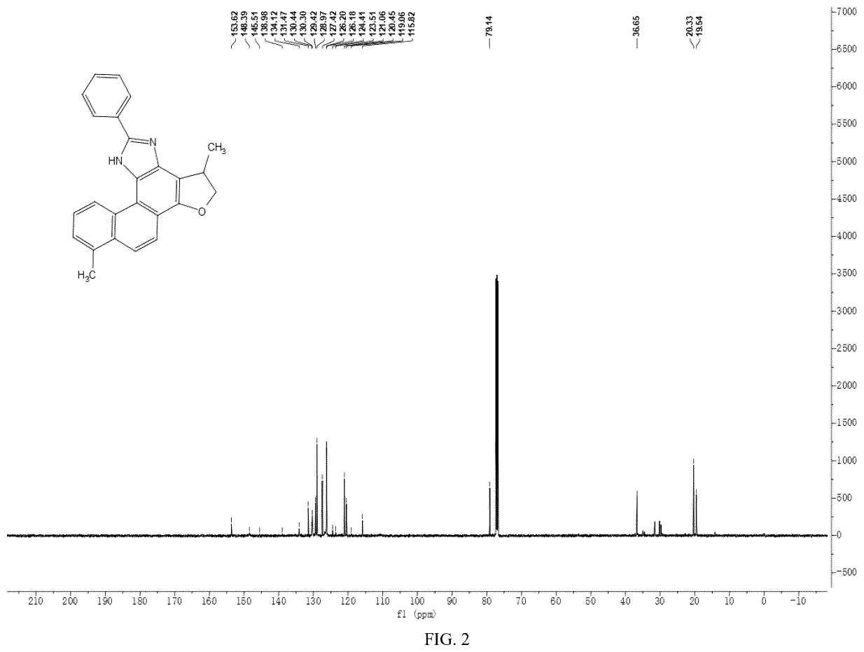

Preparation of the dihydrotanshinone I derivative: The dihydrotanshinone I derivative was prepared through a one-step reaction with dihydrotanshinone I (Shanghai Yuanye Biotechnology Co., Ltd., B20357), benzaldehyde (Macklin, B802738), and ammonium acetate (Macklin, A800996) as reactants. A 1H NMR spectrum of DPDH was shown in FIG. 1, and a ¹³C NMR spectrum of DPDH was shown in FIG. 2.

Cited reference: Li, M. M.; Xia, F.; Li, C. J.; Xu, G.; Qin, H. B. Design, Synthesis and Cytotoxicity of Nitrogen-Containing Tanshinone Derivatives. Tetrahedron Lett. 2018, 59, 46-48.
Example 2: 10 g of a dihydrotanshinone I derivative was taken, a starch was added, and granulation was conducted to produce a granule.
Example 3: 10 g of a dihydrotanshinone I derivative was taken, dextrin was added, and encapsulation was conducted to produce a capsule.
Example 4: 10 g of a dihydrotanshinone I derivative was taken, and dimethyl sulfoxide (DMSO), TWEEN80, and normal saline (8%:4%:88%) were added to prepare an injection.
Example 5: Investigation of a pharmacological effect of a dihydrotanshinone I derivative for CCM in mice
1. Animals

Cerebral cavernous malformation (CCM) 1 abbreviated as CCM1. 10 SPF-grade Ccm1^{ﬂ/ﬂ}mice and 30 Ccm1^{ECKO} mice: which were donated by Peter Choi from the Century Institute of Australia.
2. Dihydrotanshinone I derivative: which was prepared according to the method in Example 1.
3. Main reagents

Tamoxifen (Sigma-Aldrich, H7904), 4% paraformaldehyde (Biosharp, 70071800), H&E staining kit (Beyotime, C0105S), and iodine solution (Sigma-Aldrich, L6146).
4. Main instruments

Stereo microscope (Leica), pathology imaging system (PerkinElmer), and CT imaging system (PerkinElmer).
5. Animal grouping

The 10 Ccm1^{ﬂ/ﬂ}mice were adopted as a normal control. The 30 Ccm1^{ECKO} mice were randomly divided into a model group (n=10), a dihydrotanshinone I derivative group (5 mg/kg, n=10), and a dihydrotanshinone I derivative group (10 mg/kg, n=10).
6. Establishment of an animal model

Tamoxifen was intragastrically injected to establish a CCM mouse model. Specific steps were as follows: On day 1 after birth (D1), infant mice each were intragastrically injected with 50 μL of tamoxifen (0.5 mg/mL). On D19, brain tissues were collected from the mice and subjected to pharmacodynamic evaluation.
7. Therapeutic method

In the drug treatment groups, infant mice each were intraperitoneally administered with DPDH at 5 mg/kg or 10 mg/kg on day 11 after birth (D11), and then administered once every other day, specifically on D13, D15, and D17. In the normal control group and the model group, the same volume of a drug solvent was intraperitoneally injected, with 4 times in total.
8. Index detection

The overall lesions of CCMs were observed with the stereo microscope. The area of brain tissue lesions was investigated through H&E staining. The lesion counts and volumes were quantified by the CT imaging system. The vascular structure at a lesion site was detected by the scanning electron microscopy.
9. Statistical method

All data was expressed as Mean±SD. Experimental data was subjected to intergroup difference analysis with the GraphPad Prism 9 software. P<0.05 indicates a significant difference.
10. Result conclusion

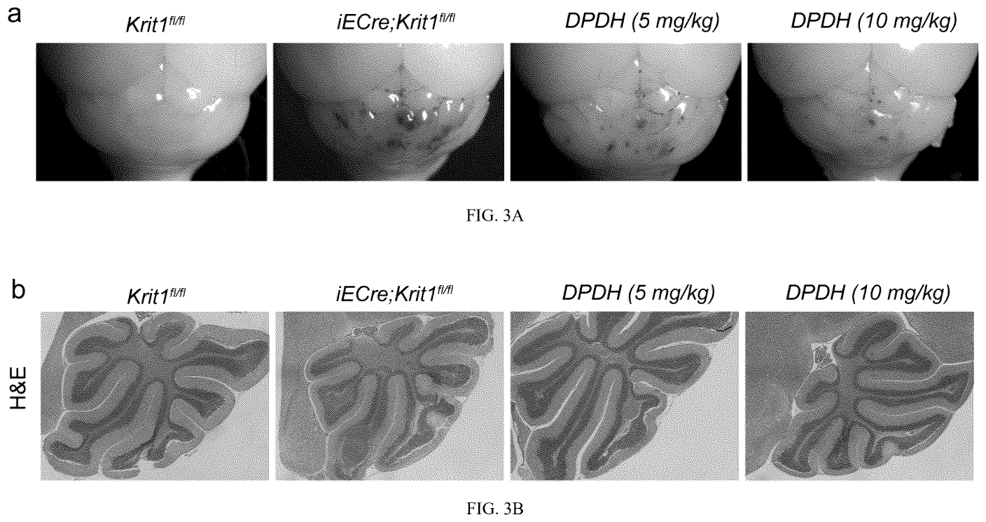

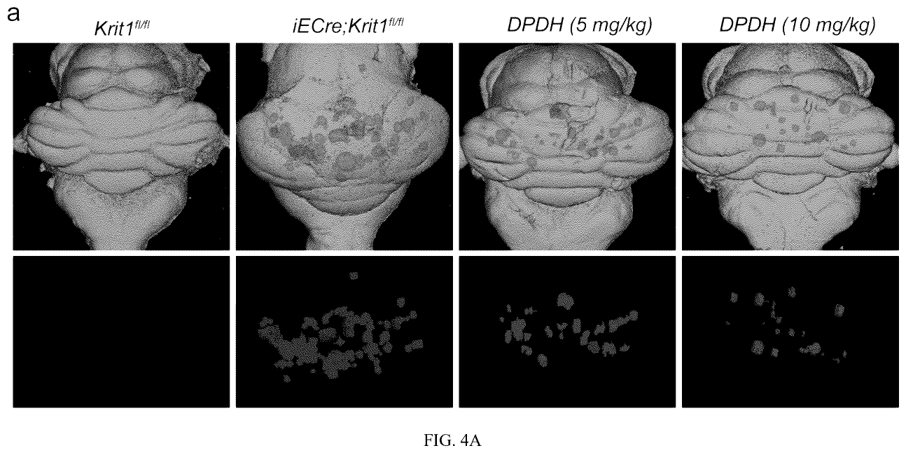

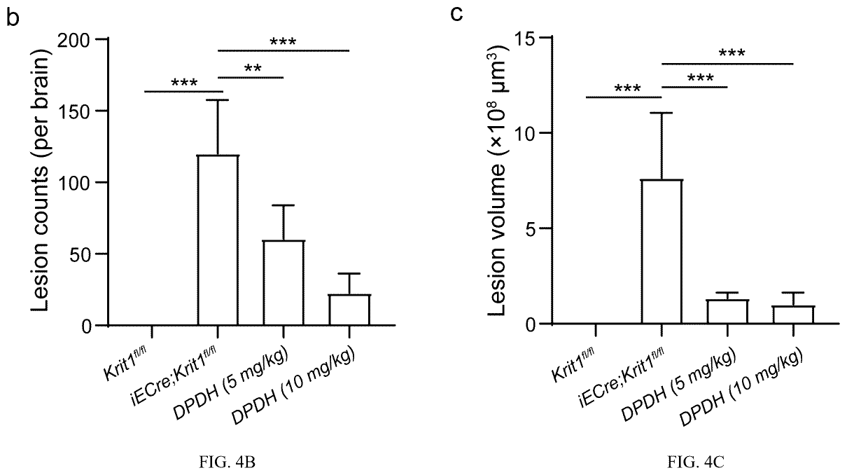

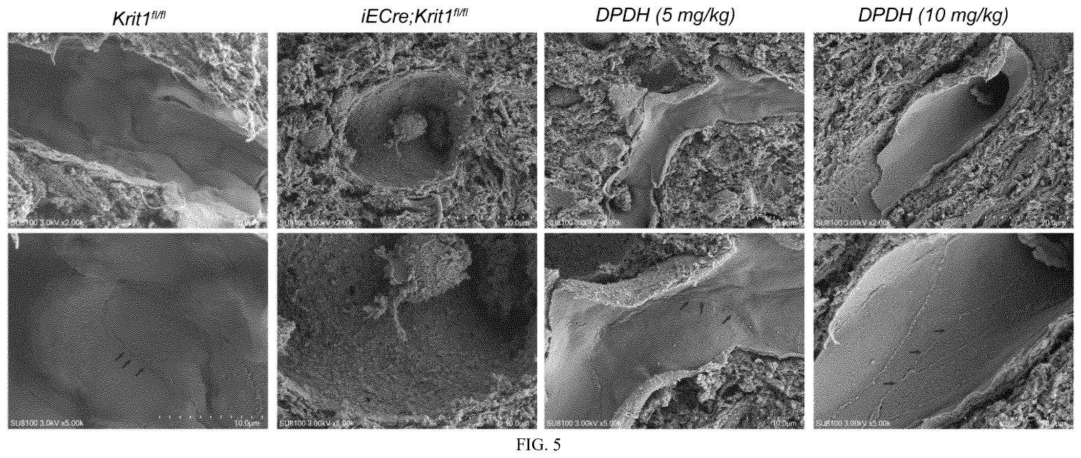

According to results, DPDH (10 mg/kg) can improve the CCM in Ccm1^{ECKO} mice (as shown in FIGS. 3A-3B). Specifically, DPDH can reduce the lesion counts and volumes (as shown in FIGS. 4A-4C) and can also alleviate the vascular structures of CCMs in Ccm1^{ECKO} mice (as shown in FIG. 5).

Conclusion: DPDH can play an anti-CCM role.

Although the embodiments of the present disclosure have been illustrated and described, it should be understood by a person of ordinary skill in the art that various changes, modifications, replacements, and variations may be made to the embodiments without departing from the principle and spirit of the present disclosure. The scope of the present disclosure is subject to the appended claims and equivalents thereof.

What is claimed is:

1. A method for treating a cerebral cavernous malformation (CCM), comprising administering a dihydrotanshinone I derivative to a subject, wherein the dihydrotanshinone I derivative has a chemical name of (R)-1,6-dimethyl-11-phenyl-1,10-dihydro-2H-furo[2′,3′:1,2]phenanthro[3,4-d]imidazole, a molecular formula of C₂₅H₂₀ON₂, and a structural formula as follows:

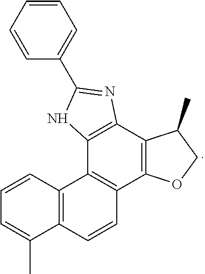 [SMILES: Cc1cccc2c1ccc1c3c(c4nc(-c5ccccc5)[nH]c4c12)[C@@H](C)CO3]

2. The method according to claim 1, wherein the dihydrotanshinone I derivative and a pharmaceutically-acceptable adjuvant are prepared into one of an oral formulation and an injection.

3. The method according to claim 2, wherein the oral formulation is one of a tablet, a hard capsule, a soft capsule, and a granule.
